(19) United States
(12) Patent Application Publication
(10) Pub. No.: US 2023/0079779 A1
(43) Pub. Date: Mar. 16, 2023

(54) CANNABIS EXTRACT AND A METHOD FOR MAKING THE SAME

(71) Applicant: Ryan Bates Bavoux, Billerica, MA (US)

(72) Inventor: Ryan Bates Bavoux, Billerica, MA (US)

(21) Appl. No.: 17/812,458

(22) Filed: Jul. 14, 2022

Related U.S. Application Data
(60) Provisional application No. 63/245,100, filed on Sep. 16, 2021.

(51) Int. Cl. A23L 33/105; B30B 15/06; B30B 15/34; A61Q 17/00

(52) U.S. Cl. CPC A23L 33/105; A61Q 17/005; B30B 15/34; B30B 15/064; A61K 2236/31; A61K 2236/37

(57) ABSTRACT

A method of producing a mechanically extracted pseudo-polymorphic Cannabis extract by compressing a Cannabis substance between a heat resistant material and placing the sandwiched Cannabis substance on a press plate, pressing the Cannabis substance at a predetermined pressure for a predetermined time with the press at a temperature, releasing the press, heating the press plates to a predetermined temperature, pressing the Cannabis substance at a predetermined pressure for a predetermined time while the press plates are being heated to the predetermined temperature, collecting an extract expressed from the Cannabis substance, and storing the extract.

### CROSS-REFERENCE TO RELATED APPLICATION

This application claims the benefit of priority of U.S. Provisional Application No. 63/245,100, filed Sep. 16, 2021, the contents of which are herein incorporated by reference.

### BACKGROUND OF THE INVENTION

The present invention relates to Cannabis extract and, more particularly, to a solventless Cannabis extract and a method for making said extract.

Bacteria and fungi are gaining resistance through mutations, to known products or compounds that prevent growth and/or cause cellular death of said bacteria and/or fungi. Bacteria and fungi are known to cause a plethora of negative effects in many industries including health, food products, agriculture, etc. There is a constant need to find new compounds that utilize various chemical pathways to eliminate or prevent the growth of bacterial and/or fungal cells by broad spectrum or narrow spectrum approach.

Current systems or products do not cause specified compound interactions with bacterial and fungal cell membranes or cells in general, that result in cell death or prevent growth. The narrow spectrum approach targets specific bacterial or fungal strains, as to allow the survival of pro-bacteria, or helpful fungal or bacterial strains. Other systems or devices need to increase the efficacy of impacts on bacteria and fungus as time goes on. New compounds need to be constantly derived to continually stay effective as general or narrow spectrum bacterial and/or fungal preventative agents, or to cause cell death. Bacteria and fungi mutate over time and increase their resistance to already used products and systems. It is beneficial to kill bacteria and/or fungi by using a targeted narrow spectrum approach, in order to allow helpful species to survive and reproduce.

The system of antimicrobial production of compounds we have today fails to make use of lineages of pseudo-polymorphic interacting compounds in extant strains, genetically stored strains, and newly formed strains of Cannabis. The specific compounds in different Cannabis strains extracted at various temperatures and pressures as an interacting group of compounds are not used alone or part of a formulation in any antimicrobial or antifungal products or treatments.

As can be seen, there is a need for pseudo-polymorphic antimicrobial sets of compounds extracted from Cannabis strains, in order to battle fungal and bacterial strains and cells that may interact with humans or pets to cause infection, disease, or illness, act as a disease preventative agent, and to target specific bacteria, fungi, or virus to limit or cease their interaction with people, food, plants, or pets.

### SUMMARY OF THE INVENTION

In one aspect of the present invention, a method of producing a mechanically extracted pseudo-polymorphic Cannabis extract comprises compressing a Cannabis substance between a heat resistant material and placing the sandwiched Cannabis substance on a press plate, pressing the Cannabis substance at a predetermined pressure ranging from 0.1 to 90 tons for a predetermined time ranging from 1-600 seconds with the press at a temperature between 1-200° F., releasing the press, heating the press plates to a predetermined temperature ranging between 50-350° F., pressing the Cannabis substance at a predetermined pressure ranging from 0.1 to 90 tons for a predetermined time ranging from 1-600 seconds while the press plates are being heated to the predetermined temperature ranging from 50-350° F., collecting an extract expressed from the Cannabis substance, and storing the extract.

These and other features, aspects and advantages of the present invention will become better understood with reference to the following drawings, description, and claims.

### BRIEF DESCRIPTION OF THE DRAWINGS

FIG. 1 is a schematic view of a press used according to an embodiment of the present invention; and

FIG. 2 is a flow chart detailing steps of a pressurized method of extracting Cannabis components according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims with reference to the drawings.

A general overview of the various features of the invention will be provided, with a detailed description following. Broadly, an embodiment of the present invention provides a pseudo-polymorphic antimicrobial set of compounds mechanically extracted from a Cannabis substance such as with a press, without use of a chemical additives or an added solvent. The compounds may be extracted and collected from the Cannabis species by means of a heat press. The Cannabis substance may be Cannabis sativa, Cannabis indica, and/or any Cannabis species or plant of the Cannabis lineage or any Cannabis that has been processed. The present invention may be used as an antibacterial, antiviral, antiparasitic, antifungal agent alone or may be added to an existing antifungal, antimicrobial, antiviral, and antiparasitic treatment/product to increase efficacy. Chemicals such as alcohols, ketones, phenols, or chemicals with hydroxyl groups that are in a stereochemical configuration enabling them to interact with other compounds, may also be used to increase the efficacy of the present invention. Treatments/products may include but are not limited to fungicides or miticides, chemicals, antivirals, antiparasitics, cancer treatments or other known antibacterial products such as Neosporin™ or penicillin.

The following method steps according to some embodiments of the present invention may be performed. Advantageously, the method steps do not include an introduction of a solvent. Instead, they produce a solventless Cannabis extract. Any Cannabis substance or processed Cannabis may be placed in a filtered pouch, a micron bag, or micron filter bag. The following discussion and examples discuss the use of a micron bag. These examples are not limiting as any bag or pouch with a surface of microscopic holes or apertures may be used. The apertures may be about 25-micron in diameter. The bag may help separate plant matter of the Cannabis substance and an extract expressed from the substance upon pressing. Different filters or micron bag pore size values may be utilized to extract different pseudo-polymorphic compounds with different capabilities, from the same Cannabis strains’ cannabinoid and terpenoid profile. Said different pseudo-polymorphic compounds may have different interactions with bacterial and fungal cells. The Cannabis substance is not required to be placed in a micron bag. The present invention may be utilized without a micron bag.

The Cannabis substance, while inside of a micron bag if used, may be placed between two sheets of a press substrate. The press substrate may be wax paper, parchment paper, or a food grade paper. The Cannabis substance may then be pressed by a press for typically 0 to 5 minutes although the time is variable in this process as lesser heat for longer periods of time can achieve the same effect as more heat for less time. The type of press is not particularly limited to the present invention. Any press or pressing method may be utilized such as an expeller or screw-type press traditionally used to extract oils. The press may be heated. For example, the press may be a 2.5 ton heat press. The pressing of the Cannabis substance enables electrons to be transferred for bond formation and breakage. Using heat to degrade a carboxylic acid to a hydroxyl group is necessary to activate Cannabis and allows the hydroxyl group to accept electrons and form an ion pull to affect bioavailability and/or lipid affinity of bacterial or fungal cell membranes. Positive stereochemical inductive effects, along with the hydroxyl groups’ electronic chemical nature allow for this bacterial or fungal chemical mechanism of action to occur. The pressing of the Cannabis substance prior to extraction without heat, enables a more uniform flow of compounds away from the center of mass through the micron filter bag without bursting the bag and contaminating the sample.

The transferring of electrons for bond formation and breakage is explained in theory below. An evolutionarily developed cross resistance is naturally formed through mutations and interaction with chemical products over evolutionary time. Therefore, Cannabis strains have the unique ability to combine with other known antibiotics to cause different interactions with bacteria (that have not been evolutionarily blocked). The hydroxyl groups on CBG, as well as the relative abundance and placement of an n-pentyl(C₅ H₁ ₁ chain), allows for change in lipid affinity. Bioavailability of ions is modified by the relative solubilities of the bacterial membrane and therefore loss of vital function for the cells’ integrity. The use of heat to degrade carboxylic acid to a hydroxyl group typically occurs to form cannabinoids. The addition of these extra hydroxyl groups adds electronegative dipoles that increase aqueous solubility of the molecules allowing for ions to be attracted to the polar hydroxyl groups while maintaining the position of the molecule with a resonating series of steroid like ring structures that are intermingling in pseudo-polymorphic arrangements. The resonating electron densities of the phenolic ring structures create a mechanism of electronic movement and Van der-Waals interactions along with the relative dipoles in position to one another, that fluctuate the electrons in space. The greater the fluctuation of electrons in space around a circular path, the greater the electromagnetic field produced by jumping to ground state configurations. This forms an ion pull along with inductive effects of the stable ring structure and the hydroxyl oxygen electronegativity. Hydroxide ions are not a good leaving group as a strong base, and therefore are more likely to accept ions. Since the oxygen is more electronegative than the hydrogen, it becomes an electron withdrawing group allowing for the lone pair to be readily donated to an acid. Positive inductive effects of long carbon chains and resonating benzene structures attached to a highly electronegative atom with a hydrogen attached add electron density of the structure toward the electronegative oxygen atom. The combination of these electron density interactions makes the lone pairs on oxygen in the hydroxyl groups attached extremely reactive. Adding phenyl groups decreases the amount of bioavailability since there are stable carbons with a double bond to hold electrons in a stable arrangement that is nonpolar.

Press plates of the press may be released or separated enough to cease pressurization of the Cannabis substance. A temperature of the press plates may then be increased to an approximate range spanning from 50-350° F. for the extraction of cannabinoids and terpenoids. The terpenes may function as a chemically active carrier and dissolving liquid for the cannabinoids. The Cannabis substance may be suspended/miscible in a solution of terpenes. The solvent terpenes are naturally occurring in suitable ratios for extraction to happen within the Cannabis substance and extra terpenoids are not added for extraction purposes.

Pressure may be slowly added to the Cannabis substance during the pressing process. This may help prevent lipid contamination of the extract, for example by preventing a micron bag blowout. It may also provide a more uniform pressure application.

As the press plates reach a maximum temperature, a maximum pressure may be applied to the Cannabis substance. This iteration of the pressing may last a maximum of approximately 600 seconds for a substantially complete extraction.

Additional, successive presses of the same Cannabis substance at varying temperatures and pressures may enable an extraction of additional and potentially different pseudo-polymorphic compounds. Successive presses may require replacing the press substrate.

Once the extraction is substantially complete or no other compounds are able to be extracted, the micron bag and Cannabis substance may be discarded. Compounds remaining on the press substrate may then be collected and stored. Storage would ideally be in an airtight container and at a constant temperature ranging from about 40-70° F. with minimal light exposure and low humidity. This storage may minimize contamination and possible inconsistencies in compound viability and composition over time.

Cannabis compound viability can be maintained while keeping the relative consistency of the compounds using the above method steps of the present invention. Oxidation and breakage of bonds of compounds may also be avoided by following the method steps.

Extracted compounds may be homogenized at temperatures lower than 200° F. in sterile glass containers. The extracted compounds may be used alone as an antibacterial, antifungal, antiparasitic, and antiviral product. Alternatively, they may be reacted with chemicals such as alcohols, ketones, phenols, or compounds that have active hydroxyl groups or reactive oxygen species that can participate in chemical reaction mechanisms or blends of these chemicals in different ratios to form stronger antibacterial, antiviral, antiparasitic, or antifungal products/interactions or improve the efficacy of the products. Specific examples include methanol, ethanol, isopropanol, methyl alcohol, ethyl alcohol, propanyl, propanol, isopropyl alcohol, 1-butanol, 2-propanol, acetone, phenylethanone, t-butyl phenyl ketone, distilled water, sodium hydroxide, DMSO, propyl alcohol. These chemicals are then diluted with various volumes of water to get different concentrations. The following chemical reaction scheme represents an example of a reaction mechanism post mechanical solventless extraction with the pseudo-polymorphic Cannabis extract: 0.25 mL of 0.19 g/mL (230° F., 850 psi Pineapple express extract dissolved in anhydrous ethanol) mixed with 2.5 mL of 0.20 g/mL (220° F., 850 psi pineapple express extract dissolved in anhydrous ethanol). A 0.5 mL aliquot of this mixture is mixed with 0.33 mL DI H₂O. After this, 2 to 200 microliters of anhydrous ethanol or isopropanol or a mixture of both, are added to the mixture. This reaction may be carried out at temperatures between 32-90° F. environmental air temperature; although optimal temperatures to carry out this reaction are between 40-70° F. Stainless steel or glass tools that have been heat sterilized can then be used to manipulate the compounds into respective containers to ensure compound composition stays the same. Manipulation may include separating aliquots, stainless steel that is heat sterilized may stop contamination of different pseudo-polymorph extracts to ensure quality control.

In some embodiments of the present invention, the extra may be pressed with press plates heated to a predetermined temperature ranging from 50-350° F. at a predetermined pressure ranging from 0.1 to 90 tons and pressed for a time ranging from 1-600 seconds to crash out the cannabinoids from the terpenoids in the extract and change bond structure.

In some embodiments of the present invention, the extract may be reacted with a solution selected from a group consisting of primary alcohols, secondary alcohols, tertiary alcohols, ketones, phenols, chemicals with active hydroxyl groups, and combinations thereof to improve the efficacy of the chemical and to improve the efficacy of the Cannabis solution as an antibacterial, antifungal, antiparasitic, and antiviral product. The reactions may include methanol, ethanol, methyl alcohol, ethyl alcohol, propanyl, propanol, isopropyl alcohol, isopropanol, 1-butanol, 2-propanol, acetone, phenylethanone, t-butyl phenyl ketone, distilled dihydrogen monoxide, sodium hydroxide, DMSO, propyl alcohol.

A product may be formed by mixing the extract with chemicals selected from a group consisting of: Cannabis extract, terpenoids, distilled water, primary alcohols, secondary alcohols, tertiary alcohols, ketones, phenols, chemicals with active hydroxyl groups, and combinations thereof.

The product may be administered by a mode selected from a group consisting of: an encapsulated pill, orally as a tablet or lozenge or troche or aqueous preparation or powder or alcohol preparation, intramuscularly, rectally, intravenously, intraarterial, intrathecal, transdermally or as an extended release patch or as an ointment or foam, inhalation, sublingually, buccally, vaginally and penially, subdermally, and subcutaneous injection.

The product may be added to a mixture selected from a group consisting of: an antibiotic compound, an antifungal compound, an antiviral compound, an antiparasitic compound, penicillin, polymyxin B, bacitracin, neomycin, cephalosporins, tetracyclines, glycopeptides, nitroimidazoles, macrolides, beta-lactams, clindamycin, metronidazole, quinolones, fluoroquinolones, aminoglycosides, sulfa biotics, sulfonamides, amoxicillin, cephalexin, ciprofloxacin, azithromycin metronidazole, chloramphenicol, clindamycin, metronidazole, vancomycin, isoniazid, rifampin, ethambutol, pyrazinamide, halogenated compounds, heavy metals, biguanides, iodophors, peroxides, hydrogen peroxide, benzoyl peroxide, phenols, hexachlorophene, triclosan, chlorine bleach, quaternary ammonium compounds, aldehydes, formaldehyde, ethylene oxide, silver nitrate, organomercurials, amphotericin B, echinocandins, flucytosine, azoles, nystatin, griseofulvin, terbinafine, alcohols, amantadine, oseltamivir, peramivir, rimantadine, ribavirin, cidofovir, foscarnet sodium, ganciclovir, valganciclovir, acyclovir, docosanol, famciclovir, idoxuridine, penciclovir, trifluridine, valacyclovir, and combinations thereof.

The product may be added to a viscous jelly media and petroleum jelly to act as an inactive carrier.

In some embodiments of the present invention alcohols, ketones, or compounds that have active hydroxyl groups or reactive oxygen species participate in chemical reaction mechanisms. This may include methanol, ethanol, Isopropanol, 1-butanol, 2-propanol, acetone, phenylethanone, t-butyl phenyl ketone, distilled water, sodium hydroxide, DMSO, propyl alcohol. These are just some examples for illustrative purposes. In said reactions, an electronegative dipole may form that will pull ions to interact with cell membranes. The following example is given for illustrative purposes: 0.25 mL of 0.19 g/mL (dissolved in anhydrous ethanol) 230° F. pineapple express extract mixed with 2.5 mL of 0.20 g/mL (dissolved in anhydrous ethanol) 220° F. pineapple express extract. A 0.5 mL aliquot of this mixture is added to 0.33 mL DI H₂ O and 20 microliters of anhydrous ethanol. The strain, extract of that strain may vary and be treated at a specific temperature and pressure with an amount of distilled water and chemicals in different ratios. The pseudo-polymorph is miscible in ethanol and reacts to form an aqueous solution. Starting concentrations may be made with ethanol or other alcohol.

The species or strain of Cannabis used, the pressure applied, the length of time the pressure is applied, and the amount of heat applied, the ratio of cannabinoids to terpenes to chemicals to DI water, the temperatures the reaction may be carried out under, the filter bag pore size used, and the order of addition of cannabinoids to terpenes to chemicals to DI water in the reaction scheme in the above method steps may vary. Variations may produce different extracts and isolate different compounds for different antibacterial, antiviral, antiparasitic, and antifungal products or interactions, as well as varying the amount of charged pseudo-polymorph species in solution. A final product may be a pseudo-polymorphic variation of a Cannabis strains’ cannabinoids and terpenoids with inter-chemical bondings, enabling different interactions between the compounds that will affect different bacteria, fungi, and viruses at varied magnitudes. Due to the different interactions, various levels of inhibition of growth, disinfection, and death of bacterial cells, fungal cells, or viral cells can be observed and used in or as products to target specified bacterial or fungal or viral cells or cell lines. The stereochemical orientation of different Cannabis compounds in space forms different pseudo-polymorphic Cannabis cannabinoid and terpenoid bonding combinations which can be identified and classified for production purposes. Each bacterial or fungal strain, infection, and/or species can be matched with a pseudo-polymorphic Cannabis extract or solution with chemicals and water described in this paper, that produces the optimal antimicrobial inhibition response toward said bacteria, fungi, or virus. The extract may be a combination of both cannabinoids and terpenoids together.

A final product may inhibit growth and potentially kill bacterial and fungal cells or cell lines, as well as viral cells. They may also stop infection from bacteria and fungi. These pseudo-polymorph extracts can be used alone, or they can be mixed with other known antibacterial, antifungal, or antiviral compounds to form more effective products to avoid cross resistance mechanisms that form throughout evolutionary time.

Each pseudo-polymorph may also be used by themselves or in a chemical solution described above or can be used as a topical treatment. The final product may be used to treat disease or infection via an encapsulated pill, orally as a tablet or lozenge or troche or aqueous preparation or powder or alcohol preparation, intramuscularly, rectally, intravenously, intraarterial, intrathecal, transdermally or as an extended release patch or as an ointment or foam, inhalation, sublingually, buccally, vaginally and penially, subdermally, and subcutaneous injection.

The final product can be used as an agricultural pesticide in chemical solution or as a topical antimicrobial or antifungal agent. This product can be used as a preservative, or preservation mechanism for substances that are susceptible to bacteria and fungi such as food products or drugs. The present invention can be used in the healthcare industry for various applications with a plethora of effects in many areas such as topical use for wounds, allergic reactions, skin conditions or diseases, infections of all kinds, cancer therapy or treatment supplement, immune system supplement etc.

The final product may be used as an antibacterial, antifungal, antimicrobial, antiparasitic, therapeutic, anticancer, supplemental therapy to cancer treatment, immunotherapy, oncology immunotherapy, and even anti-anxiety/stress medication. Crystal lattice structures can also be formed by the knockout of terpenes with pressure and heat induced by a successive press of only the Cannabis extract; of which may have a plethora of potential various effects on bacteria and fungi.

In some embodiments of the present invention approximately 61.2% alcohol can be added to 0.20 g/mL Cannabis pseudo-polymorph extract which contains both terpenes and cannabinoids, followed by the addition of deionized water at approximately 39.7% of the solution volume. Other alcohols, ketones, phenols, or chemicals described herein may be used in addition to a chemical and Cannabis solution to change the chemical entourage effects between the compounds in solution and in turn, their reactions with respective bacterial and/or fungal targets. Any chemical compound with an active hydroxyl functional group or oxygen that participates in chemical mechanisms can be used to increase the efficacy of the bactericidal interaction. This includes methanol, ethanol, methyl alcohol, ethyl alcohol, propanyl, propanol, isopropyl alcohol, isopropanol, 1-butanol, 2-propanol, acetone, phenylethanone, t-butyl phenyl ketone, distilled dihydrogen monoxide, sodium hydroxide, and dimethyl sulfoxide.

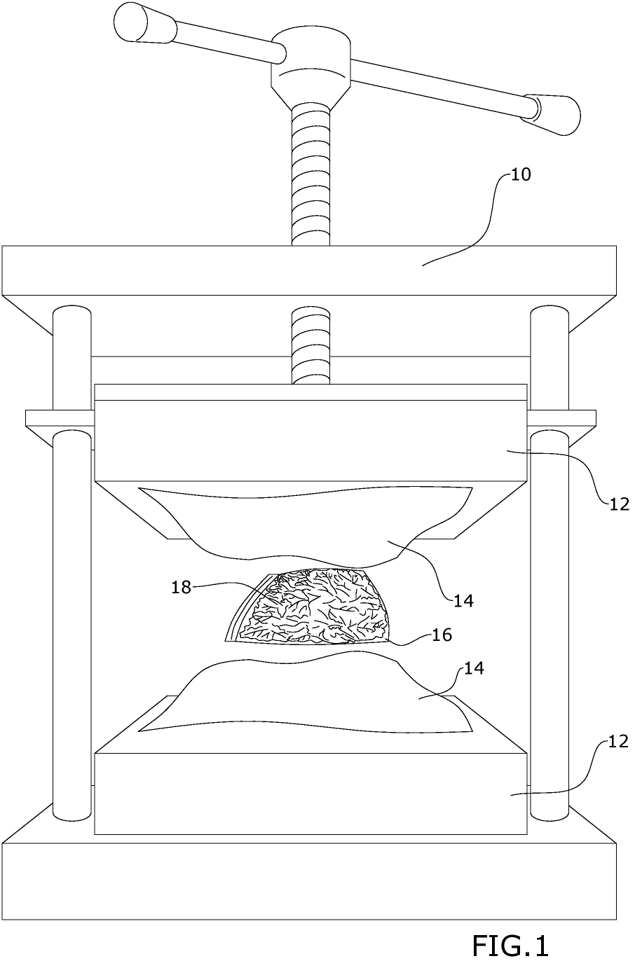

Referring now to the Figures, FIG. 1 is a schematic view of a press 10 according to an embodiment of the present invention. The press comprises heat plates 12. A Cannabis substance 18 is placed in a micron filter bag 16. The micron filter bag 16 is positioned between sheets of parchment paper 14 between the heat plates 12. The press 10 may press the Cannabis substance 18 with the heat plates 12 to extract pseudo-polymorphs.

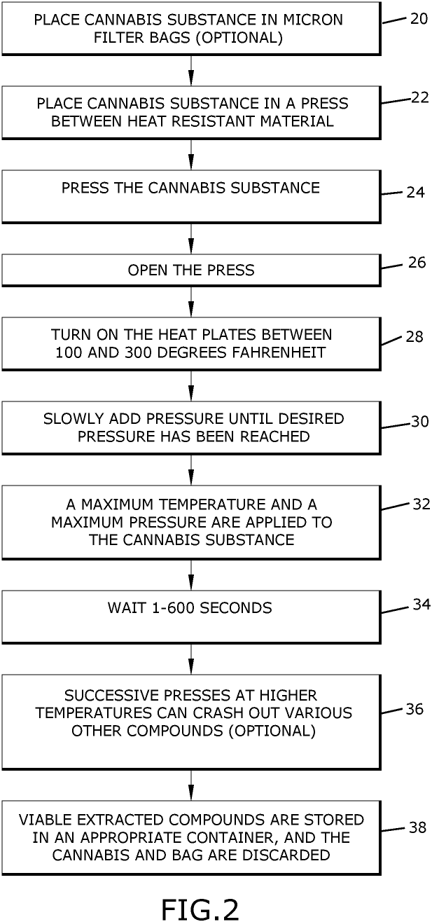

FIG. 2 is a flow chart detailing method steps according to an embodiment of the present invention. A Cannabis substance, placed in a micron filter bag 20 if applicable, is compressed or sandwiched between any heat resistant material 22, such as food grade parchment paper. The Cannabis substance 18 is pressed 24 at a predetermined pressure for a predetermined period. This pressing may last between 0-600 seconds, for example. The press is then opened or released 26. Press plates may then be heated to a predetermined temperature, such as between 50-350° F. 28. While the plates are heating or after the plates have been heated, the Cannabis substance may be pressed again at any rate, however a full compression within 30 seconds is most optimal. A slow press of the Cannabis substance helps ensure terpenes will not be knocked out of the cannabinoid crystalline structure. A maximum temperature and maximum pressure of the press may be applied simultaneously 32. Upon reaching a maximum temperature and maximum pressure, the Cannabis substance is continually pressed for 1-600 seconds 34 The excreted Cannabis extract may be collected 38 at this point. Alternatively, successive presses may be applied 36 before collection 38 of the extract. If successive presses are applied, the heat resistant material may be replaced at each iteration of a successive press. Upon collection 38 of the extract, the Cannabis substance and micron bag are discarded. In addition, the Cannabis extract can be sequentially pressed to form crashed out lattice formations from the terpenoid dissolvent/carrier.

It should be understood, of course, that the foregoing relates to exemplary embodiments of the invention and that modifications may be made without departing from the spirit and scope of the invention as set forth in the following claims.

What is claimed is:

1. A method of producing a mechanically extracted pseudo-polymorphic Cannabis extract comprising:
compressing a Cannabis substance between a heat resistant material and placing the sandwiched Cannabis substance on a press plate;
pressing the Cannabis substance at a predetermined pressure ranging from 0.1 to 90 tons for a predetermined time ranging from 1-600 seconds with the press at a temperature between 1-200° F.;
releasing the press;
heating the press plates to a predetermined temperature ranging between 50-350° F.;
pressing the Cannabis substance at a predetermined pressure ranging from 0.1 to 90 tons for a predetermined time ranging from 1-600 seconds while the press plates are being heated to the predetermined temperature ranging from 50-350° F.;
collecting an extract expressed from the Cannabis substance; and
storing the extract.

2. The method of claim 1, further comprising placing the Cannabis substance into a micron filter bag before placing the Cannabis substance on the press plate.

3. The method of claim 1, further comprising reacting the extract with a solution selected from a group consisting of: primary alcohols, secondary alcohols, tertiary alcohols, ketones, phenols, chemicals with active hydroxyl groups, and combinations thereof.

4. The method of claim 1, wherein terpenes are extracted from the Cannabis substance and act as an active carrier for the extract.

5. The method of claim 1, further comprising pressing the extract with press plates heated to a predetermined temperature ranging from 50-350° F. at a predetermined pressure ranging from 0.1 to 90 tons and pressing a time ranging from 1-600 seconds.

6. The method of claim 1, further comprising forming a product by mixing the extract with a chemical selected from a group consisting of: Cannabis extract, terpenoids, distilled water, primary alcohols, secondary alcohols, tertiary alcohols, ketones, phenols, chemicals with active hydroxyl groups, and combinations thereof.

7. The method of claim 6, further comprising administering the product topically to an infection.

8. The method of claim 6, further comprising administering the product as a food preservative.

9. The method of claim 6, further comprising administering the product by a mode selected from a group consisting of: an encapsulated pill, orally as a tablet or lozenge or troche or aqueous preparation or powder or alcohol preparation, intramuscularly, rectally, intravenously, intraarterial, intrathecal, transdermally or as an extended release patch or as an ointment or foam, inhalation, sublingually, buccally, vaginally and penially, subdermally, and subcutaneous injection.

10. The method of claim 6, further comprising adding the product to mixture selected from a group consisting of: an antibiotic compound, an antifungal compound, an antiviral compound, an antiparasitic compound, penicillin, polymyxin B, bacitracin, neomycin, cephalosporins, tetracyclines, glycopeptides, nitroimidazoles, macrolides, beta-lactams, clindamycin, metronidazole, quinolones, fluoroquinolones, aminoglycosides, sulfa biotics, sulfonamides, amoxicillin, cephalexin, ciprofloxacin, azithromycin metronidazole, chloramphenicol, clindamycin, metronidazole, vancomycin, isoniazid, rifampin, ethambutol, pyrazinamide, halogenated compounds, heavy metals, biguanides, iodophors, peroxides, hydrogen peroxide, benzoyl peroxide, phenols, hexachlorophene, triclosan, chlorine bleach, quaternary ammonium compounds, aldehydes, formaldehyde, ethylene oxide, silver nitrate, organomercurials, amphotericin B, echinocandins, flucytosine, azoles, nystatin, griseofulvin, terbinafine, alcohols, amantadine, oseltamivir, peramivir, rimantadine, ribavirin, cidofovir, foscarnet sodium, ganciclovir, valganciclovir, acyclovir, docosanol, famciclovir, idoxuridine, penciclovir, trifluridine, valacyclovir, and combinations thereof.

11. The method of claim 6, further comprising adding the product to a viscous jelly media and petroleum jelly.
